(19) United States
(12) Patent Application Publication
(10) Pub. No.: US 2010/0240775 A1
(43) Pub. Date: Sep. 23, 2010

(54) PASTEURISATION STABLE STARCH COMPOSITIONS

(76) Inventors: Bernd Wolfgang Kettlitz, Bonheiden (BE); Jozef Victor Jean Marie Coppin, Denderleeuw (BE); Stephane Jules Jerome Debon, Denderleeuw (BE); Florent Francois Laurent Meert, Erembodegem (BE)

Correspondence Address: FISH & RICHARDSON P.C., PO BOX 1022, MINNEAPOLIS, MN, 55440-1022, US

(21) Appl. No.: 12/295,102

(22) PCT Filed: Mar. 21, 2007
(86) PCT No.: PCT/EP2007/052684

§ 371 Date: Sep. 29, 2008

(30) Foreign Application Priority Data
Mar. 31, 2006 (EP) 06251802.2

(51) Int. Cl. A61K 47/36; A23L 1/0522

(52) U.S. Cl. 514/778; 426/578

(57) ABSTRACT

The present invention relates to novel compositions having (a) at least one pregelatinized starch and (b) at least one cook-up starch or flour, and which develop upon mixing with water a low cold viscosity, develop no or a low viscosity during the pasteurisation step and develop a significantly higher viscosity at a further heating step.

### TECHNICAL FIELD

The present invention is in the field of starch-based compositions which exhibit no or low viscosity development during the pasteurisation step.

### BACKGROUND OF THE INVENTION

Pasteurisation is an important process in many applications involving starch-based products. The pasteurisation process is commonly carried out by heating. A good heat penetration in aqueous solutions/suspensions during the pasteurisation step is thereby important. The development of viscosity at temperatures between 65° C. and 85° C. substantially hinders an effective heat penetration and therefore an effective pasteurisation. Starch-based products are commonly employed in the food industry to provide the important viscosity in products, for example in sauces and soups. Conventional starch-based products develop substantial viscosities at pasteurisation temperatures between 65° C. and 85° C. Therefore, a reheating of such starch-based products after pasteurisation does not lead to the desired increase in viscosity necessary for the good mouthfeel. Efforts have been undertaken to develop starch-based products that exhibit significant viscosities only above the pasteurisation temperature. Generally, osmotic pressure increasing agents and water-activity depressing agents have been employed to achieve these goals.

EP 0 012 465 reports a way of reducing the rate of swelling of starch in an aqueous starch concentrate during pasteurisation. This is achieved by lowering the osmotic gradient between the interior of the starch granule and the aqueous solution in which the starch is dispersed. The osmotic gradient is lowered by adding suitable compounds such as salts, glycerol, glucose and protein hydrolysates to the concentrate. The addition of hot water to the starch concentrate leads to the decrease in the concentration of these compounds and this again leads to the swelling of the starch.

U.S. Pat. No. 4,291,066 reports a process for producing an aqueous concentrate which can be pasteurised and subsequently reconstituted into a lump-free, thick soup, sauce, gravy or dessert. The process involves producing a concentrate comprising (i) a proportion of starch sufficient to obtain a product of the desired degree of creaminess, (ii) a proportion of an osmotic pressure increasing agent, sufficient to substantially reduce the rate of swelling of starch granules, and (iii) a proportion of a water-activity depressing agent so that the water-activity is not exceeding 0.92.

There is still a need for starch-based compositions which exhibit moderate viscosity at ambient temperature and no or a low viscosity increase at pasteurisation temperatures between 65 and 85° C. The present invention addresses this need by providing such starch-based compositions without having to resort to osmotic pressure increasing and/or water-activity depressing agents.

### SUMMARY OF THE INVENTION

The present invention relates to a composition that comprises or consist essentially of (a) at least one pregelatinized starch and (b) at least one cook-up starch or flour, wherein the cook-up starch or flour has an amylose content of between 10 and 40% by weight based on the dry substance of the starch or flour. If the cook-up starch or flour is native, then the number average starch granule particle size of the cook-up starch or flour is equal or less than 20 micrometer. In embodiment of the invention, the ratio by weight on dry basis of the two components, (a) and (b), is from 1:0.5 to 1:3. In another embodiment, the ratio by weight on dry basis of the two components, (a) and (b), more preferably 1:1 to 1:1.5.

The invention further relates to a liquid composition, a food composition, a soup and sauce formulation comprising the composition according to the invention. Furthermore, the invention also refers to the use of the composition according to the present invention in food, feed, cosmetic, or pharmaceutical applications, in particular where the application involves a pasteurisation step at 65° C. to 85° C., preferably 65° C. to 75° C.

### DETAILED DESCRIPTION OF THE INVENTION

“Pregelatinized starches”—also known as pregelled or instant starches—means starches which yield a viscous paste when dispersed in cold water. Swollen granules in pregelatinised starches may be predominantly intact, e.g. in spray-cooked starches, or partially intact, e.g. in roll-dried starches.

The term “cook-up starch” includes any granular starch that requires water and heat in order to gelatinize and paste. It includes both native and modified starches.

“Modified starch” means any starch which has been treated chemically or physically so as to change the functionality of the starch and to modify one or more of its key physical or chemical properties such as thickening properties, freeze-thaw stability, gel formation, cold water solubility, shear resistant viscosity, and the like. Physically modified starches for example include but are not limited to thermally-inhibited starches.

“Flour” means foodstuffs obtained from the meal of a grain or other starchy food sources. Commonly, flours are powders made from cereal grains or other starchy food sources and which are still containing proteins, such as for example wheat gluten.

The term “native starch” means starch recovered in the original form by extraction from any starch-bearing material.

The number average starch granule particle size is measured by laser diffraction with a Sympatec laser diffraction sensor HELOS/BF.

The amylose content of the starch is determined according to a standard method as described in Morrison, W. R, Laignelet, B., Journal of Cereal Science, 1, 9-20, 1983. The subject matter of which is incorporated by reference.

The water activities were measured on an Aqualab CX-2 from Wilten Instruments at 20° C.

“Cross-bonded starch” within this context means starch which has been treated with a bi- or polyfunctional reagent so that a small number of the starch polymer chains are chemically linked by the cross linking reagent moiety.

“Substituted starch” means starch where substituent groups have been introduced at the hydroxyl groups of the glucose units. Substitution of starches may be via esterification or etherification of the glucose units.

In a first aspect, the present invention relates to a composition which comprises or consists essentially of (a) at least one pregelatinized starch and (b) at least one cook-up starch or flour, whereby the cook-up starch or flour has an amylose content of between 10 and 40% by weight on the dry substance of the starch or flour, preferably between 10% and 30%, more preferably between 15% and 25%. The cook-up starch of the composition may be modified or native starch. If the cook-up starch or flour is native, then the number average starch granule particle size of said native starch or flour has to be equal or less than 20 micrometer, preferably less than 10 micrometer, more preferably less than 5 micrometer.

This invention is based in part on the surprising findings of the present inventors that when heating aqueous slurries of the composition of the present invention to pasteurisation temperatures between 65° C. to 85° C., no or a low viscosity development can be observed. In a particular instance, surprising findings were observed at pasteurisation temperatures of from about 65° C. to 75° with respect to low viscosity development. Such a low viscosity development is not expected because in commonly used starch-based compositions the viscosity at any particular temperature is equal to the sum of the viscosities of each starch component when measured separately. Additionally, it has been found that this low viscosity development has no negative effect on the development of the full viscosity at temperatures above 90° C.

The surprising effect of low viscosity development at pasteurisation temperature is illustrated in Example 1 where the viscosity development of a slurry of a starch-based composition containing waxy rice starch is compared with the viscosity development of a slurry of a composition containing rice starch. Both slurries contain the same amount of a cold water soluble spray-cooked starch (2.8% w/w) which gives a Brabender viscosity of about 200 BU at 50° C. for both slurries. The concentration of the waxy rice starch and the rice starch was kept the same in both slurries at 3% w/w. Both slurries were heated according to the same temperature profile on a Micro Visco-Amylo Graph® by Brabender®. In order to determine the viscosity development, the Brabender viscosities were measured at 95° C. and 70° C. during the heating and the ratio of the viscosities (Brabender viscosity at 95° C./Brabender viscosity at 70° C.) calculated. “During the heating” means that the Brabender viscosities are measured first at 70° C. and then after further heating at 95° C. without any cooling step in between. This ratio gives an indication on one hand of the viscosity development at pasteurisation temperatures between 65° C. to 85° C., and on the other hand an indication of the ability to develop full viscosity at higher temperatures. The much higher ratio of the Brabender viscosities at 95° C. and 70° C. (Brabender viscosity at 95° C./Brabender viscosity at 70° C.) for the slurry containing the rice starch (4.1) than for the slurry containing the waxy rice starch (1.2) indicates that surprisingly only rice starch is pasteurisation stable at temperatures between 65° C. to 85° C. but not waxy rice starch. Further investigations showed that surprisingly only slurries containing compositions comprising (a) at least one pregelatinized starch and (b) at least one cook-up starch or flour, characterized in that the cook-up starch or flour has an amylose content of between 10% and 40% by weight based on the dry substance of the starch or flour, and wherein, in case that said cook-up starch or flour is native, the number average starch granule particle size of the cook-up starch or flour is equal or less than 20 micrometer, can provide both functions; the functions being, pasteurisation stability between 65° C. to 85° C. and full viscosity development at higher temperatures. Additionally, a slurry containing a composition according to the present invention has the advantage that the cold water viscosity at 50° C. is on one hand big enough to avoid phase separation and precipitation of food ingredients in the slurry, and on the other hand small enough to allow efficient heat transfer within the liquid. Heat transfer is particularly important for the pasteurisation step.

The surprising findings by the present inventors are based in part on the development of a novel method for measuring the viscosity profiles of starches on a Micro Visco-Amylo Graph® by Brabender®. Commonly, the measurements of starch viscosity profiles on a Micro Visco-Amylo Graph® by Brabender® are carried out on a 5.5% w/w starch slurry in water at a pH of 5.5. A problem with this setup is that the gelatinisation temperature of the starch measured does not correspond to the actual gelatinisation temperature and it is therefore not possible to select pasteurisation stable starches from such measurements. Through research in how to improve the measurement methods, it was found that the gelatinisation temperature of starches is more accurately determined by measuring the Brabender viscosity temperature profile of a starch in an aqueous solution of pH 4 to 8 or alternatively, pH 5 to 7, and having a Brabender viscosity at 50° C. in the range between 100 and 500 BU, preferably in the range between 100 and 400 BU, more preferably in the range between 100 and 300 BU and most preferably in the range between 150 and 200 BU. These findings were confirmed by differential scanning calorimetry and microscopy. It was found that regular maize starch starts to swell in an aqueous solution at 65° C., whereas no such swelling was found up to 70° C. when standard methods were used. “Standard methods” in this context mean any method for measuring the Brabender viscosity/temperature dependence in an aqueous solution having no significant viscosity at 50° C. This new method therefore provides crucial information for selecting starches according to their granule swelling characteristics.

In one embodiment of the present invention, the ratio by weight on dry basis of components (a) and (b) of the composition of the present invention is from 1:0.5 to 1:3, preferably 1:1 to 1:1.5. Furthermore, the composition preferably comprises 33 to 75% of cook-up starch or flour (component (b)) by weight based on the dry substance of the composition.

The first component of the composition according to the present invention is a pregelatinized starch (component (a)). The pregelatinized starch may be both a modified or an unmodified starch, or a mixture of both. Preferred modified pregelatinized starches are n-octenyl succinate starch, cross-bonded-hydroxypropylated starch, cross-bonded-acetylated starch, and mixtures thereof. Preferred unmodified pregelatinized starches are waxy maize starch, regular maize starch, potato starch, tapioca starch, and mixtures thereof. In a particular aspect of the present invention, the pregelatinized starches are made by spray-cooking.

Component (b) of the composition according to the present invention may be a cook-up starch or a flour. The cook-up starch in the composition may be native or modified. Preferred native starches as cook-up starches in the present invention are rice starch, wheat B starch, wheat starch, quinoa starch, sorghum starch, regular maize starch, saponaria vaccaria starch, and mixtures thereof. In a particular embodiment of the invention, compontent (b) is rice starch, wheat B starch, or quinoa starch. Preferred modified starches as cook-up starches in the present invention are cross-bonded, cross-bonded substituted starch, and mixtures thereof. In a particular embodiment of the invention, modified starches are acetylated di-starch adipates and hydroxypropylated di-starch phosphates made from starches selected from the group consisting of regular maize starch, tapioca starch, wheat starch, and mixtures thereof.

The preferred flour to use as component (b) in the composition of the present invention is rice flour.

The addition of emulsifiers may be capable of increasing the ratio of the Brabender viscosities of the compositions in aqueous solution measured at 95° C. and 70° C. during the heating process. Preferred emulsifiers are anionic such as diacetyl tartaric acid ester of mono-diglycerides (DATEM) and sodium stearoyl lactylate (SSL).

The compositions according to the present invention may be in the form of a dry mix, for example as a powder, or in any other suitable form. This invention relates to a dry mix comprising the composition according to the current invention. The present invention also relates to a liquid composition comprising the composition of the present invention and an edible liquid. Preferred liquid compositions comprise 2% to 7% w/w of the composition of the present invention, preferably from 3% to 5%.

The compositions of the present invention are particularly of interest in food, feed, cosmetic, and pharmaceutical applications, especially whenever there is a requirement for a pasteurisation step and the development of significantly higher viscosity at a further heating step. The invention therefore further relates to a food composition comprising the composition of the present invention and a further food ingredient. Food ingredients include but are not limited to vegetable pieces, fruits, raisins, meat, pasta pieces, and the like. The food composition may be employed in powder form, as a liquid or slurry or in any other suitable form.

The compositions of the present invention are particularly advantageous in the preparation of soups and sauces. Food ingredients present in soups and sauces, for example vegetable or pasta pieces, do not precipitate and agglomerate together at cold temperatures due to the presence of a cold water viscosity. Preferred soup or sauce formulations comprise 2% to 7% w/w the composition of the present invention, preferably from 3% to 5%.

No or low viscosity development of starches can generally be achieved by adding osmotic pressure increasing agents and/or water-activity depressing agents to starch slurries. It has to be understood that it is the object of this invention to provide pasteurisation stable starch-based compositions that do not require such additions. The slurries and other applications comprising the compositions according to the present invention can therefore be characterized that they have a water-activity of more than 0.95, preferably more than 0.97.

The current invention is illustrated in the following examples.

### Example 1

The Brabender viscosity measurements were carried out on a Micro Visco-Amylo-Graph® by Brabender®. Slurries of (A) 3% rice starch (Remy FG obtained from Remy Industries, Wijgmaal, Belgium) and (B) 3% waxy rice starch (Remy FG obtained from Remy Industries, Wijgmaal, Belgium) by weight on dry basis and 2.8% spray-cooked stabilised n-octenyl succinate waxy maize starch (C*Em Tex 06328 obtained by Cargill, prepared as described in WO 2005/100407 A1) weight on dry basis were prepared with water. The total weight of the slurry was 105 g. The pH was adjusted to 5.5 with 0.1 N NaOH or 0.1 N HCl.

The starch-water slurry was then stirred at 250 rpm and heated according to the following temperature profile:
starting at 50° C.
rising to 68° C. at a rate of 3° C./min
remaining at 68° C. for 105 s
rising to 95° C. at a rate of 3° C./min
cooling to 50° C. at a rate of 3° C./min.

TABLE 1
Brabender viscosities at 50° C. and ratios of the Brabender viscosities
at 95° C. and 70° C. of compositions (A) and (B) (Brabender viscosity
at 95° C./Brabender viscosity at 70° C.) measured according to the
above method during the heating process.

| | | | Brabender | |
|---|---|---|---|---|
| Cook-up | | Brabender | viscosity at 95° C./ | Water |
| starch/ | Pregelatinized | viscosity | Brabender | activity |
| flour | starch | at 50° C. | viscosity at 70° C. | (a_{w}) |
| Rice | C*Em Tex | 200 | 4.1 | 0.99 |
| starch | 06328 | | | |
| Waxy rice | C*Em Tex | 200 | 1.2 | 0.99 |
| starch | 06328 | | | |

### Example 2

A slurry of 3% wheat B starch (obtained from Cargill Wheat Plant at Barby, Germany) by weight on dry basis and 2.8% spray-cooked stabilised n-octenyl succinate waxy maize starch (C*Em Tex 06328 obtained by Cargill, prepared as described in WO 2005/100407 A1) weight on dry basis was prepared with water. The total weight of the slurry was 105 g. The pH was adjusted to 5.5 with 0.1 N NaOH or 0.1 N HCl.

TABLE 2
Brabender viscosity at 50° C. and ratio of the Brabender viscosities
at 95° C. and 70° C. (Brabender viscosity at 95° C./Brabender
viscosity at 70° C.) measured according to the method in Example 1.

| | | | Brabender | |
|---|---|---|---|---|
| Cook-up | | Brabender | viscosity at 95° C./ | Water |
| starch/ | Pregelatinized | viscosity | Brabender | activity |
| flour | starch | at 50° C. | viscosity at 70° C. | (a_{w}) |
| Wheat B | C*Em Tex | 200 | 3.5 | 0.99 |
| | 06328 | | | |

### Example 3

A slurry of 3% rice flour (obtained from Cargill Wheat Plant at Barby, Germany) by weight on dry basis and 2.8% spray-cooked stabilised n-octenyl succinate waxy maize starch (C*Em Tex 06328 obtained by Cargill, prepared as described in WO 2005/100407 A1) weight on dry basis was prepared with water. The total weight of the slurry was 105 g. The pH was adjusted to 5.5 with 0.1 N NaOH or 0.1 N HCl.

TABLE 3
Brabender viscosity at 50° C. and ratio of the Brabender viscosities
at 95° C. and 70° C. (Brabender viscosity at 95° C./Brabender
viscosity at 70° C.) measured according to the method in Example 1.

| | | | Brabender | |
|---|---|---|---|---|
| Cook-up | | Brabender | viscosity at 95° C./ | Water |
| starch/ | Pregelatinized | viscosity | Brabender | activity |
| flour | starch | at 50° C. | viscosity at 70° C. | (a_{w}) |
| Rice flour | C*Em Tex | 200 | 4.9 | 0.99 |
| | 06328 | | | |

### Example 4

A slurry of 3% rice flour (obtained from Oriental Food Co. Ltd., Bangkok, Thailand, Art. no. 13261) by weight on dry basis, 2.8% spray-cooked stabilised n-octenyl succinate waxy maize starch (C*Em Tex 06328 obtained by Cargill, prepared as described in WO 2005/100407 A1) weight on dry basis and 0.075% Panodan M2010 DATEM emulsifier (obtained from Danisco) by weight on dry basis was prepared with water. The total weight of the slurry was 105 g. The pH was adjusted to 5.5 with 0.1 N NaOH or 0.1 N HCl.

TABLE 4
Brabender viscosity at 50° C. and ratio of the Brabender viscosities at 95° C. and
70° C. (Brabender viscosity at 95° C./Brabender viscosity at 70° C.)
measured according to the method in Example 1.

| | | | | Brabender | |
|---|---|---|---|---|---|
| | | | | viscosity at | |
| Cook-up | | | Brabender | 95° C./Brabender | Water |
| starch/ | Pregelatinized | | viscosity at | viscosity at | activity |
| flour | starch | Emulsifier | 50° C. | 70° C. | (a_{w}) |
| Rice | C*Em Tex | Panodan M2010 | 200 | 6.7 | 0.99 |
| flour | 06328 | DATEM | | | |
| | | (diacetyl tartaric | | | |
| | | acid ester of | | | |
| | | mono- | | | |
| | | diglycerides) | | | |

### Example 5

A slurry of 3% rice flour (obtained from Oriental Food Co. Ltd., Bangkok, Thailand, Art. no. 13261) by weight on dry basis, 2.8% spray-cooked stabilised n-octenyl succinate waxy maize starch (C*Em Tex 06328 obtained by Cargill, prepared as described in WO 2005/100407 A1) weight on dry basis and 0.075% Grindsted SSL (sodium stearoyl lactylate) P 55 VEG (obtained from Danisco) by weight on dry basis was prepared with water. The total weight of the slurry was 105 g. The pH was adjusted to 5.5 with 0.1 N NaOH or 0.1 N HCl.

TABLE 5
Ratio of the Brabender viscosities of the above composition at 95° C. and 70° C.
(Brabender viscosity at 95° C./Brabender viscosity at 70° C.)
measured according to the method in Example 1.

| | | | | Brabender | |
|---|---|---|---|---|---|
| | | | | viscosity at | |
| Cook-up | | | Brabender | 95° C./Brabender | Water |
| starch/ | Pregelatinized | | viscosity at | viscosity at | activity |
| flour | starch | Emulsifier | 50° C. | 70° C. | (a_{w}) |
| Rice | C*Em Tex | Grindsted SSL | 200 | 5.5 | 0.99 |
| flour | 06328 | (sodium | | | |
| | | stearoyl | | | |
| | | lactylate) P 55 | | | |
| | | VEG | | | |

### Example 6

A slurry of 3% high cross bonded regular maize adipate by weight on dry basis, 2.8% spray cooked stabilised n-octenyl succinate waxy maize starch (C*Em Tex 06328 obtained by Cargill, prepared as described in WO 2005/1004 007 A1) weight on dry basis and 0.075% Panodan M2010 DATEM emulsifier (obtained from Danisco) by weight on dry basis was prepared with water. The total weight of the slurry was 105 g. The pH was adjusted to 5.5 with 0.1 N NaOH or 0.1 N HCl 0.1N.

TABLE 6
Ratio of the Brabender viscosities of the above composition at 95° C. and 70° C.
(Brabender viscosity at 95° C./Brabender viscosity at 70° C.)
measured according to the method in Example 1.

| | | | | Brabender | |
|---|---|---|---|---|---|
| | | | | viscosity at | |
| Cook-up | | | Brabender | 95° C./Brabender | Water |
| starch/ | Pregelatinized | | viscosity at | viscosity at | activity |
| flour | starch | Emulsifier | 50° C. | 70° C. | (a_{w}) |
| high | C*Em Tex | Panodan M2010 | 200 | 6.4 | 0.99 |
| cross | 06328 | DATEM | | | |
| bonded | | (diacetyl tartaric | | | |
| regular | | acid ester of | | | |
| maize | | mono- | | | |
| adipate | | diglycerides) | | | |

### Example 7

A slurry of 3% high cross bonded regular maize adipate by weight on dry basis, 2.8% spray cooked stabilised n-octenyl succinate waxy maize starch (C*Em Tex 06328 obtained by Cargill, prepared as described in WO 2005/1004 007 A1) weight on dry basis and 0.075% Grindsted SSL (sodium stearoyl lactylate) P 55 VEG (obtained from Danisco) by weight on dry basis was prepared with water. The total weight of the slurry was 105 g. The pH was adjusted to 5.5 with 0.1 N NaOH or 0.1 N HCl 0.1N.

TABLE 7
Ratio of the Brabender viscosities of the above composition at 95° C. and 70° C.
(Brabender viscosity at 95° C./Brabender viscosity at 70° C.)
measured according to the method in Example 1.

| | | | | Brabender | |
|---|---|---|---|---|---|
| | | | | viscosity at | |
| Cook-up | | | Brabender | 95° C./Brabender | Water |
| starch/ | Pregelatinized | | viscosity at | viscosity at | activity |
| flour | starch | Emulsifier | 50° C. | 70° C. | (a_{w}) |
| high | C*Em Tex | Grindsted SSL | 200 | 6.0 | 0.99 |
| cross | 06328 | (sodium | | | |
| bonded | | stearoyl | | | |
| regular | | lactylate) P 55 | | | |
| maize | | VEG | | | |
| adipate | | | | | |

### Example 8

The following soup formulation was prepared:

A slurry of 1.5% high cross bonded regular maize adipate by weight on dry basis, 2.8% spray-cooked stabilised n-octenyl succinate waxy maize starch (C*Em Tex 06328, prepared as described in WO 2005/100407 A1) by weight on dry basis, 11.5% tomato puree (32% dry substance) by weight, 1.2% skimmed milk powder by weight, 0.5% table salt by weight and 1% sucrose by weight was prepared with water. The total weight of the slurry was 105 g. The ratio of the Brabender viscosities of the soup at 95° C. and 70° C. (Brabender viscosity at 95° C./Brabender viscosity at 70° C.) measured during the heating process is shown in Table 8.

TABLE 8
Ratio of the Brabender viscosities of the above soup formulation
at 95° C. and 70° C. (Brabender viscosity at
95° C./Brabender viscosity at 70° C.) measured
according to the method in Example 1.

| | | | Brabender | |
|---|---|---|---|---|
| Cook-up | | Brabender | viscosity at 95° C./ | Water |
| starch/ | Pregelatinized | viscosity | Brabender | activity |
| flour | starch | at 50° C. | viscosity at 70° C. | (a_{w}) |
| high cross | C*Em Tex | 200 | 2.8 | 0.99 |
| bonded | 06328 | | | |
| regular | | | | |
| maize | | | | |
| adipate | | | | |

1. A composition comprising (a) at least one pregelatinized starch and (b) at least one cook-up starch or flour, characterized in that the cook-up starch or flour has an amylose content of between 10 and 40% by weight based on the dry substance of the starch or flour, and wherein, when said cook-up starch or flour is native, the average starch granule particle size of the cook-up starch or flour is equal or less than 20 micrometer.

2. The composition according to claim 1 wherein the ratio by weight on dry basis of component (a) to component (b) is from 1:0.5 to 1:3.

3. The composition according to claim 1 wherein the composition comprises 33% to 75% of cook-up starch or flour by weight based on the dry substance of the composition.

4. The composition according to claim 1 wherein the cook-up starch is a native starch selected from the group consisting of rice starch, wheat B starch, wheat starch, quinoa starch, sorghum starch, regular maize starch, saponaria vaccaria starch, and mixtures thereof.

5. The composition according to claim 1 wherein the cook-up starch is a modified starch and is selected from the group of cross-bonded, cross-bonded substituted starch, and mixtures thereof.

6. The composition according to claim 5 wherein the modified starch is an acetylated di-starch adipate starch or hydroxypropylated di-starch phosphate starch selected from the group consisting of regular maize starch, tapioca starch, wheat starch, and mixtures thereof.

7. The composition according to claim 1 wherein the flour is rice flour.

8. The composition according to claim 1 wherein the pregelatinized starch is selected from the group consisting of modified pregelatinized starch, unmodified pregelatinized starch, and mixtures thereof.

9. The composition according to claim 8 wherein the pregelatinized starch is a modified starch and is selected from the group consisting of n-octenyl succinate starch, cross-bonded-hydroxypropylated starch, cross-bonded-acetylated starch, and mixtures thereof.

10. The composition according to claim 9 wherein the pregelatinized starch is an unmodified pregelatinized starch and is selected from the group consisting of a waxy maize starch, regular maize starch, potato starch, tapioca starch, and mixtures thereof.

11. The composition according to claim 9 wherein the pregelatinized starch is spray-cooked starch.

12. Dry mixes comprising the composition according to claim 1 and an edible liquid.

13. A liquid composition comprising the composition according to claim 1 on an edible liquid.

14. The liquid composition according to claim 13 wherein the composition according to claim 1 is present from 2% to 7% by weight of the liquid composition.

15. The liquid composition according to claim 14 wherein the water activity is more than 0.95.

16. A food composition comprising the composition according to claim 1 and a further food ingredient.

17. A soup or sauce formulation comprising the composition according to claim 1.

18. The coup or sauce formulation according to claim 17 wherein the composition according to claim 1 is present from 2% to 7% by weight of the soup or sauce formulation.

19. The soup or sauce formulation according to claim 18 wherein the water activity is more than 0.95.

20. A food, feed, cosmetic, or pharmaceutical application comprising a composition according to claim 1 or the dry mix according to claim 12.

21. The food, feed, cosmetic, or pharmaceutical application according to claim 20 wherein said food, feed, cosmetic, or pharmaceutical application is pasteurized at 65 to 85° C.

22. The food, feed, cosmetic, or pharmaceutical application according to claim 20, wherein said food, feed, cosmetic, or pharmaceutical application is pasteurized at 65 to 75° C.
